(19) United States
(12) Patent Application Publication
(10) Pub. No.: US 2023/0248633 A1
(43) Pub. Date: Aug. 10, 2023

(54) DELIVERY OF COSMETIC AGENTS, COMPOSITIONS AND USE THEREOF

(71) Applicant: ELC MANAGEMENT LLC, Melville, NY (US)

(72) Inventors: Snehal M. SHAH, Nesconset, NY (US); DANIELA BRATESCU, Northport, NY (US); Isaac David COHEN, Brooklyn, NY (US); Zhihan ZHOU, Metuchen, NJ (US); Mary Ann SMAIL, Commack, NY (US); Milan Franz SOJKA, Coram, NY (US); Joe H. COLAS, West Babylon, NY (US); Peter A. LANDA, Springfield, NJ (US)

(21) Appl. No.: 18/302,726

(22) Filed: Apr. 18, 2023

Related U.S. Application Data
(62) Division of application No. 17/187,882, filed on Mar. 1, 2021.
(60) Provisional application No. 62/984,168, filed on Mar. 2, 2020.

(51) Int. Cl. A61K 8/73; A61K 8/49; A61Q 19/08

(52) U.S. Cl. CPC A61K 8/731; A61K 8/4953; A61Q 19/08; A61K 2800/413; A61K 2800/805; A61K 2800/412

(57) ABSTRACT

The present invention provides novel methods for repairing or reducing skin changes as well as systems, regimens, and cosmetic compositions thereof. The invention relates to a cosmetic composition comprising electrospun polymer fibers, at least one active ingredient, and at least one cosmetically acceptable carrier is provided, wherein the electrospun fibers are dispersed in the composition. The composition is in form of a solution, suspension, lotion, cream, gel, emulsion, toner, ointment, paste, foam, hydrogel, film-forming product, or facial skin mask. The composition comprises at least one active ingredient and further comprises antioxidants, moisturizers, surfactants, or humectants.

### FIELD

The present invention generally relates to methods, systems, and cosmetic or dermatologic compositions for repairing and reducing skin defects using polymeric fibers, including microfibers and nanofibers.

### BACKGROUND

Aging, exposure to adverse environmental factors, pollutants, lack of good nutrition, fatigue, can affect the visual appearance, physical properties, or physiological functions of the skin. These factors may create a visually undesirable appearance to the skin. Notable changes on the skin include, for example, changes in the eye area such as dilation of the veins, bags under the eyes, dark circles under the eyes, and swelling around the eyes. Changes in other areas of the face include, for example, fine lines and wrinkles, loss of elasticity, loss of stiffness, loss of uniformity of color or tone, rough surface texture, age spots, and a decrease in moisture content. Many of such changes in the appearance and function of the skin are caused by changes in the outer epidermal layer of the skin, while other changes are caused by changes in the lower dermis.

Polymer-based nanofibers are used as an implanted drug delivery vehicle in pharmaceutical industries. However, due to its high cost and low productivity, the application of such material in cosmetic products is limited. Further, with the development of electrospinning technology, the production cost and scale-up capabilities have significantly improved in recent years.

It is therefore an object of the present invention to utilize polymeric microfibers or nanofibers for cosmetic delivery systems, compositions, and use thereof.

### SUMMARY

One aspect of the invention relates to a cosmetic composition comprising electrospun polymer fibers, at least one active ingredient, and at least one cosmetically acceptable carrier is provided, wherein the electrospun fibers are dispersed in the composition. The composition is in form of a solution, suspension, lotion, cream, gel, emulsion, suspension, toner, ointment, paste, foam, hydrogel, film-forming product, or facial skin mask. The composition comprises at least one active ingredient selected from a group consisting of cosmetic agents, peptides, DNA, RNA, polymers, proteins, vitamins, organic acids, enzymes, oils, and mixtures thereof. The composition further comprises antioxidants, moisturizers, surfactants, or humectants. The electrospun polymeric hydrophobic fibers are selected from the group consisting of polycarbothane, polyvinyl acetate, polysulfone, polyvinyl chloride, polylactide (PLA), polyethylene, polystyrene, polyvinylchloride, polytetrafluorethylene, polydimethylsiloxane, polyurethanes, polylactic acid, polytetrafluoroethylene, cellulose acetate, and mixtures thereof. The fibers are present from about 0.01% to about 10% by weight, relative to the total weight of the composition and further comprises a diameter of about 0.5 nm to about 0.5 μm

Another aspect of the invention relates to a method of reducing skin defects or improving the appearance of a subject's skin is provided. The method comprising topically applying to a subject's skin in need thereof, a composition comprising electrospun hydrophobic fibers, and at least one active ingredient. The method improves dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, or moisture content in the skin. The method further comprises application of the composition at least once a day, including in the morning and prior to retiring to bed. The method further comprises the use of the composition as a treatment regimen to effect a change in the skin.

Another aspect of the invention relates to a system or a kit comprising a composition comprising electrospun fibers hydrophobic fibers, at least one active ingredient, and cosmetically acceptable carriers are provided. The system or kit further includes instructions regarding use.

### BRIEF DESCRIPTION OF THE DRAWINGS

FIG. 1A and FIG. 1B show the percentage of the penetration of the AA2G from the formula from a skin model (Franz Cell) penetration study.

FIGS. 2A and 2B show the permeability of AA2G in the skin PAMPA study.

FIG. 2C shows the results of the skin penetration study beginning at 3 hours' time point.

FIG. 2D shows comparative results of the six hours penetration of AA2G between skin PAMPA study and skin penetration study results.

FIG. 3 shows penetration study of caffeine in chopped fibers formula in 24 hours (Skin Penetration (Franz Cell)).

FIGS. 4A and 4B shows the results shows the permeability of caffeine in skin PAMPA study.

FIG. 4C shows the results of the skin model penetration (Franz Cell) of caffeine with chopped fibers.

FIG. 5 shows the results of penetration of the hydrophobic active and chopped fibers in 24 hours via skin penetration study (Franz Cell).

FIG. 6 shows the penetration of the hydrophobic active obtained by the skin PAMPA study.

### DETAILED DESCRIPTION

To facilitate an understanding of this invention, several terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

The terms “active ingredient” or “active agent” or “cosmetic agent” means a cosmetic agent that is utilized to deliver a benefit to the skin. “Active ingredient” or “active agent” or “cosmetic agent” would cause to, drive a change in the subject's skin or deliver the benefits under consideration, thus, aid in accomplishing a desired, expected, or intended result. The terms “active ingredient” or “active agent” or “cosmetic agent” according to the present invention include cosmetically acceptable excipients or carriers that may be present in a composition/formulation.

The terms “prevent” and “preventing” include the prevention of the recurrence, spread or onset of a skin or hair condition. It is not intended that the present invention be limited to complete prevention.

The term “subject” refers to any mammal, preferably a human.

The term “topical” refers to the administration of an agent or agents (e.g., cosmetic, vitamin, etc.) on the skin.

The terms “transdermal” or “topical” refers to the delivery of an agent (e.g., cosmetic, dermatological, vitamin, etc.) through the skin (e.g., so that at least some portion of the population of particles reaches underlying layers of the skin).

The term “hydrophilic” refers to the physical property of a molecule that is able to transiently associate with water, i.e., bond with water via hydrogen bonding. The term “hydrophobic” refers to the physical property of a molecule that is repelled from a mass of water.

The term “solvent” refers to a liquid, solid, or gaseous solute generating a solution.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The term microfiber refers to fibers having a diameter of greater than 1000 nm.

The term nanofiber refers to fibers having a diameter of less than 1000 nm.

Except in operating and comparative examples, or where otherwise explicitly indicated, all numbers in this description indicating amounts or ratios of material or conditions of reaction, physical properties of materials and/or use are to be understood as modified by the word “about”. All amounts are presented as percentages by weight of the final cosmetic agent unless otherwise specified.

The present invention relates to methods for repairing the human skin using polymeric electrospun fibers, including microfibers and nanofibers. In some embodiments, the present invention relates to compositions for repairing the skin using polymeric electrospun nanofibers. Measurable changes appear in the skin as the skin ages or endure environmental or age-related insult. Such insults cause a general reduction in cellular and tissue vitality, reduction in cell replication rates, reduced cutaneous blood flow, reduced moisture content, errors in structure and function, alterations in biochemical pathways, and reduction of skin's ability to remodel and repair itself.

As a non-limiting example, human skin around the periorbital area (i.e., around the eyes) is thin and delicate. The periorbital area is webbed with tiny capillaries and blood sometimes leaks from these capillaries causing an appearance of dark circles under the eye. Other known causes of dark under-eye circles include UV exposure (e.g., exposure to the sun can increase natural melanin levels and draws the melanin to the surface of the skin, making it darker), ageing (e.g., with age, the skin around the eyes can become even thinner which makes dark under-eye circles become more pronounced), fatigue (being tired can make skin paler which makes dark circles look darker), allergies (e.g., allergic reactions can cause smudges in the under-eye area and conditions that causes a person to rub their eyes can make dark circles worse because scratching or rubbing can darken the skin), pregnancy or menstruation (e.g., skin becomes pale during pregnancy and menstruation which makes dark circles look darker), and inadequate nutrition.

As another non-limiting example, a condition where the skin under the eyes swells and becomes visually undesirable is called puffy eyes. Puffy eyes can be caused by several factors, including increased vascularization, leaky capillaries, thinning/slackening skin which can fill up with more fluid, loss of the fat pad under the eye which can contribute to under-eye bags, and allergies, dust, and pollutants which can trigger a release of chemicals, thereby, swelling the tissue around the eyes.

Methods for treating or repairing the skin include stimulation of the dermis or epidermis with many cosmetic active ingredients. The use of such agents may renew skin cell rate and cause basal cell division. Several approaches have been utilized to prevent, reduce or treat damage to the skin, especially eyes caused by environmental factors, chemicals, pollutants, or aging. Most of the approaches to date involve delivering one or more agents to act on the skin to cause an effect. Examples of such agents include retinoids to stimulate collagen and tools to stimulate epidermis renewal, such as films or patches that are impregnated with or carrying active agent(s) within the patch.

According to an aspect of the present invention, compositions and methods to treat the appearance of the human skin is provided, wherein such compositions comprise polymeric microfibers or nanofibers that are insoluble in water and hydrophobic in nature. Applicants of the present invention surprisingly found that the presence of polymeric nanofibers, which are insoluble and hydrophobic, in a cosmetic formulation greatly enhanced penetration of skincare active ingredients.

Soluble hydrophilic polymer nanofibers, such as PVA, have been reported to enhance the penetration of active ingredients. For example, water-soluble PVP nanofibers enhance the penetration of hydrophobic actives into the human skin. Applicants of the present invention surprisingly discovered that hydrophobic and water-insoluble polymeric nanofibers enhanced penetration of active ingredient(s).

In the art of cosmetic formulation, it is traditionally known that the use of hydrophobic substances in a formulation would hinder the cosmetic benefits and the manufacturing methods that may be utilized in process of preparing the composition. Therefore, the general approach relies on utilizing water-soluble and hydrophilic materials.

The inventors of the present application discovered that the use of hydrophobic nanofibers did not prevent the delivery of the active ingredients into the skin. On the contrary, the hydrophobic nanofibers greatly enhanced the penetration of active agents into the skin. They also discovered that the addition of water-insoluble polymeric nanofibers acted as an occlusive layer that assisted and enhanced hydration of the skin. Further, the sensorial feeling of the occlusive layer, primarily due to the soft and flexible nature of the hydrophobic nanofibers, enhanced penetration of active ingredients into the skin.

Accordingly, one aspect of the present invention comprises a topical cosmetic composition comprising electrospun hydrophobic polymeric fibers that are dispersed in the composition. The composition may also comprise hydrophilic polymeric fibers within the composition in any amount. The composition further comprises cosmetic active ingredients and carriers.

In some embodiments, the polymeric micro or nanofibers include, without limiting, polycarbothane (aliphatic, polycarbonate-based TPU), Shore A 75 through Shore D 72, poly(Vinyl Acetate), polysulfone poly(vinyl chloride), biodegradable polylactide (PLA), polyethylene, polystyrene, polyvinylchloride, polytetrafluorethylene, polydimethylsiloxane, polyesters, polyurethanes, acrylics, epoxies, polylactic acid, polytetrafluoroethylene, polyketals, cellulose acetate. In preferred embodiments, the polymeric nanofiber is utilized, including cellulose acetate.

In some embodiments, the composition may comprise electrospun hydrophilic polymeric fibers of any shape or size dispersed in the formulation. Hydrophilic polymers include, without limiting, poly(ethylene glycol), poly(propylene glycol), poly(vinyl alcohol), polypyrrolidone, or polyvinylpyrrolidone (PVP), and the biodegradable polyactive (a soft poly ethylene glycol-terephalate block copolymer with a hard poly butylene-terephthalate) among others. The polymeric fibers are electrospun fibers.

Any type of skincare active can be used and is contemplated to be within the disclosure of the present invention. The skincare actives can be hydrophobic, hydrophilic, or amphiphilic. The skincare active may be a small molecule, lipid, peptide, DNA molecules, biomolecules, enzymes, or mixtures thereof. To better disperse the polymeric nanofiber, the nanofibers are pre-cut or chopped into a predetermined length and are present in the composition as dispersed fibers, also referred to in the present disclosure as “dispersed” in the composition.

In preferred embodiments, the nanofibers are dispersed in the composition and are present as chopped fibers, having a diameter ranging from about 0.5 nm to about 5 pam. In further embodiments, the nanofibers comprise a diameter ranging from about 5 nm to 1000 nm. In preferred embodiments, the nanofibers comprise a diameter ranging from about 0.05 μm to 0.5 μm. The length of the fiber (as a dispersion, in form of chopped fibers) is about 0.1-10 mm. In some embodiment, the length is about 1-3 mm. All ranges and subranges are contemplated to be within the subject matter of the present invention. In compositions, the nanofibers are present from about 0.01% to about 10% by weight, relative to the total weight of the composition.

According to an embodiment, the cosmetic composition may comprise but is not limited to, one or more of a DNA repair enzyme, a sunscreen active, a humectant, a botanical extract, a peptide, an oil, a thickener, a surfactant, a vitamin, an antioxidant, a preservative, or a carrier. The carrier is dermatologically or cosmetically acceptable when present in the composition.

The composition may be formulated as a cosmetic product in a cosmetic carrier in form of an emulsion, cream, lotion, gel, serum, solution, spray, base, or foam.

In one embodiment, the present invention contemplates a liquid composition comprising polymeric electrospun hydrophobic micro or nanofibers for use on the skin. According to an embodiment, the composition comprising polymeric electrospun hydrophobic micro or nanofibers further comprises active ingredient, cosmetic agents, or cosmetically acceptable carriers or excipients or softeners. In one embodiment, the composition further comprises hydrophobic polymeric fibers (including microfibers or nanofibers). The polymeric fiber is porous, soft, and flexible. The hydrophobic polymeric fibers are dispersed in the composition. They may exist in the composition as a dispersion, in form of chopped fibers, of any shape, orientation, or size.

Applicants of the present invention unexpectedly and surprisingly achieved the cosmetic benefit of improving the skin's appearance using the composition disclosed herein. The composition provided significant benefits, showing a reduction in the dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin around the eyes and in the facial skin. Additional benefits obtained by the composition include, without limiting, the use of the composition as an antioxidant, collagen booster, as well as, lightening of dark spots, smoothening of wrinkles, promotion of healing, and/or reduction of scars.

The composition of the present invention may be utilized with other modes of delivery including microneedles, iontophoresis, and/or electroporation. For example, in one embodiment, microneedles are applied to the skin and the composition is applied thereafter. Any and all combinations and permutations in the use are contemplated to be part of the present invention.

The composition of the present invention may be formulated as a cosmetic product. In some embodiments, the composition can be formulated to have a pH in a range of about 1-10. In some embodiments, the compositions can be formulated to have a pH of about less than 3.0, 3.5, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, to about 12.0, or more, or any range or integer derivable therein.

The composition can be formulated as a cosmetic product in a cosmetic carrier as an emulsion, cream, lotion, gel, serum, solution, base, spray, or foam. In some embodiments, the composition may be formulated for use more than one, two, three, four times a day. In preferred embodiments, the composition may be formulated for use once, twice a day, or more. In more preferred embodiments, the composition may be formulated for use in the morning and prior to retiring to bed at night.

In compositions, the polymeric microfibers (or nanofibers) comprise a hydrophobic polymer. In further preferred embodiments, the polymeric nanofibers comprise a hydrophobic polymer, including cellulose acetate.

The composition according to the present invention is not limited by the nature of the polymer(s) used for the microfibers (or nanofibers). Any variety of polymers that are hydrophobic and water-insoluble can be used. In some embodiments, multiple (different) polymers can be used together or separately. The present invention is also not limited by the nature of the biomaterial.

In one embodiment, the composition comprising polymeric hydrophobic micro or nanofibers further includes active ingredient(s). The active ingredients may be incorporated into the formulation or may be incorporated into the fibers by being impregnated within the dispersed fibers. In some embodiments, the active ingredients may be incorporated into the fibers during electrospinning.

Another aspect of the invention is a topical delivery system. In one embodiment, the topical delivery system comprises at least one skincare active ingredient and polymeric micro or nanofibers in a cosmetic composition. The polymeric micro or nanofibers are electrospun, water-insoluble, and hydrophobic, and are present in the composition as a dispersion in form of chopped fibers. The system may further include a treatment regimen, instructions regarding how to use the composition of the present invention with any other similar cosmetic compositions or applications.

Another aspect of the present invention is the use of the compositions comprising hydrophobic electrospun polymeric micro or nanofibers for personal use, including cosmetics application by a human subject. In some embodiments, the composition may be utilized as a skincare agent. In some other embodiments, the composition may be utilized as an agent to treat or prevent conditions on the skin. In some embodiments, the composition may be utilized as a cleansing agent, exfoliating agent, or skin repairing agent.

In another aspect, a method of improving, treating, repairing, or reducing a skin's visual appearance is provided. The method comprising topically administering an effective amount of a composition comprising electrospun polymeric hydrophobic nanofibers comprising active ingredient or cosmetic agents. The present invention relates to a method for repairing or improving the skin, comprising by providing a human subject, a skin care composition according to the present invention and administering the skincare composition by applying or contacting with the subject's skin. In embodiments, the composition is in a liquid form. In further embodiments, the composition is administered topically or transdermally. In some embodiments, the composition may be applied one time or more than one time in a given day. In some embodiments, the composition may be applied at night prior to retiring to bed.

In particular, the composition may be used to prevent or treat changes to the skin, such as dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin. Particularly, the present invention relates to methods for repairing or reducing dilation of the veins, bags under the eyes, dark circles under the eyes, swelling around the eyes, fine lines and wrinkles, loss of elasticity, wrinkles, loss of stiffness, tightening, firming, loss of uniformity of color or tone, rough surface texture, age spots, and a decrease in moisture content in the skin around the eyes and in the facial skin.

In additional embodiments, the subject exhibits symptoms associated with or is suspected of having an affected visual appearance, physical properties or physiological functions of the skin, such as visually undesirable appearance to the skin, including dilation of the veins, bags under the eyes, dark circles under the eyes, and swelling around the eyes may occur, environmental damage to the skin, fine lines and wrinkles, loss of elasticity, wrinkles, loss of stiffness, loss of uniformity of color or tone, rough surface texture, age spots, and a decrease in moisture content. Topical application of the composition can treat or prevent such a skin condition. The effectiveness of the composition can be compared with skin that has not been treated or addressed with a composition of the present invention. In certain non-limiting embodiments, the skin treatment can be localized to and/or around an area (such as eyes) where the composition is applied to the skin. The skin can be a facial, torso, back, neck, ear, pelvic, arms, hands, legs (e.g., ankle, knee, thigh), feet, or buttocks skin. Non-limiting examples of skin conditions that can be treated or prevented with compositions of the present invention include telangiectasia (i.e., spider veins), eye circles (e.g., dark circles under the eye), puffy eyes, pruritus, lentigo, age spots, senile purpura, keratosis, melasma, blotches, wrinkles, fine lines, nodules, sun-damaged skin, acne, or hyperpigmentation. In certain aspects, the skin condition can be caused by exposure to UV light, age, irradiation, chronic sun exposure, environmental pollutants, air pollution, wind, cold, heat, chemicals, disease pathologies, or smoking. The skin to be treated can be aged, nutritionally compromised, or environmentally damaged skin. In certain aspects, the composition can be topically applied in an amount effective to increase the stratum corneum turnover rate of the skin, collagen synthesis production of the skin, fat production of the skin, firmness of the skin, or elasticity of the skin. In other aspects, the composition can be topically applied in an amount effective to reduce or inhibit new capillary formation in or near the skin, blood flow to the skin, the fluid amount in or near the skin, or melanin production in the skin.

In embodiments, the invention contemplates polymeric hydrophobic fibers such as cellulose acetate nanofibers, cellulose acetate microfibers, a combination of both. In preferred embodiments, the polymer fibers are chopped and dispersed in small sizes, with a diameter of about 5 nm to about 1000 nm, more preferably about 5 nm to 500 nm. The fibers may be dispersed in the composition.

Also, disclosed are systems or kits that can include a composition of the present invention. In certain non-limiting aspects, the composition is comprised in a container. The container can be a bottle, dispenser, package, etc. The container can be configured to dispense a pre-determined amount of the composition. The container can be configured to dispense the composition in a liquid, spray, emulsion, or aerosol form. In certain aspects, the system or kit can include indicial on its surface and/or instructions for using the composition.

In other aspects of the present invention, the composition can be used as part of a regimen to treat a skin condition. For instance, the regimen can include applying a composition of the present invention in a first instance as disclosed throughout this specification. The regimen can then include additional applications that are identical, similar, or different than the first instance application. The additional applications can include, for example, a second, third, fourth, fifth, sixth, seventh, eighth, nine, tenth, or more applications with a composition of the present invention and/or whether another method for treating a particular skin condition (e.g., other compositions, etc.). The regimen may also include applying the composition in the morning and/or prior to retiring to bed at night.

### Active Ingredients and Forms

According to an aspect of the present invention, the cosmetic composition may comprise but is not limited to, one or more of a DNA repair enzyme, a sunscreen active, a humectant, a botanical extract, a peptide, an oil, a thickener, a surfactant, a vitamin, an antioxidant, a preservative, or a carrier. If present suggested ranges are from about 0.0001 to 35%, preferably from about 0.0005 to 20%, more preferably from about 0.001 to 15%. The carrier is dermatologically or cosmetically acceptable when present in the composition.

In some embodiments, the composition is a liquid composition. The composition can be formulated as a cosmetic product in a cosmetic carrier as an emulsion, cream, lotion, gel, serum, solution, base, or foam.

According to an embodiment, the formulation comprising the cosmetic agent may be applied to mammalian keratinous tissue, to human skin, face or hair. The formulation comprising the cosmetic agents may be of various forms. For example, some non-limiting examples of such forms include solutions, suspensions, lotions, creams, gels, emulsions, suspension, toners, ointments, cleansing agents, exfoliating agents, liquid shampoos and hair conditioners, pastes, foams, powders, mousses, shaving creams, hydrogels, film-forming products, facial and skin masks, and the like.

The formulation type of the cosmetic agents of the present invention may be of any type, including solution system, soluble system, emulsion system, gel system, powder dispersing system, or water-oil two-phase system.

The composition may be in the form of an aqueous solution, gel, cream, lotion, emulsion, serum, spray, or suspension. The emulsion may be either water in oil or oil in water. The composition may also be anhydrous. The composition may be in the liquid, semi-solid, or solid form.

If the composition is present as an aqueous solution or dispersion, the amount of water present may range from about 0.01-99% and the amount of dissolved or dispersed solids from about 10 to 99.99%. If the composition of the invention is present in the emulsion form, it may comprise from about 0.1-99% water and from about 0.1-80% oil. If the composition of the present invention is in an anhydrous form, it may contain about 0.1-99% oil.

Conventional cosmetic adjuvants that may be suitable as additives are, for example, co-emulsifiers, fats and waxes, stabilizers, thickeners, biogenic agents, film formers, fragrances, dyes, pearlescent agents, preservatives, pigments, electrolytes (for example magnesium sulphate) and pH regulators. Co-emulsifiers are preferably known W/O and also ON emulsifiers such as polyglycerol esters, sorbitan esters, or partially esterified glycerides. Typical examples of fats are glycerides; as waxes which may be mentioned in combination with hydrophilicized len growing inter alia beeswax, paraffin wax, or microcrystalline waxes. Metal salts of fatty acids such as magnesium, aluminum and/or zinc stearate can be employed. Suitable thickeners are, for example, crosslinked polyacrylic acids and derivatives thereof, polysaccharides, more especially xanthan gum, guar-guar, agar-agar, Alginate, and tyloses, carboxymethylcellulose and hydroxyethylcellulose, and also fatty alcohols, monoglycerides and fatty acids, polyacrylates, polyvinyl alcohol and polyvinylpyrrolidone. Customary film formers are, for example, hydrocolloids such as chitosan, microcrystalline chitosan or quaternized chitosan, polyvinylpyrrolidone, vinylpyrrolidone-vinyl acetate copolymers, polymers of the acrylic acid series, quaternary cellulose derivatives, and similar compounds. Suitable preservatives are, for example, formaldehyde solution, p-hydroxybenzoate, or sorbic acid. Pearlizing agents, for example, such as ethylene glycol distearic esters come coldistearat, but also fatty acids and fatty acid into consideration. The dyes suitable for cosmetic purposes, and authorized substances may be used. Such dyes are normally used in concentrations of 0.001 to 0.1 by the weight %, based on the total mixture.

The compositions of the invention can accordingly be in liquid, paste, or solid form, for example as water-in-oil creams, oil-in-water creams, and lotions, aerosol foam creams, gels, oils, grease pencils, dusting powders, sprays, or hydroalcoholic lotions. The composition may include any active ingredient or cosmetic agent along with cosmetically acceptable excipients or carriers.

### DNA Repair Enzyme

The compositions may also contain one or more DNA repair enzymes. DNA repair enzymes may be present in a range of an amount from about 0.00001 to about 35%, preferably from about 0.00005 to about 30%, more preferably from about 0.0001 to about 25% of one or more DNA repair enzymes.

DNA repair enzymes as disclosed in U.S. Pat. Nos. 5,077,211; 5,190,762; 5,272,079; and 5,296,231, are suitable for use in the compositions described herein and method of the present invention. One example of such a DNA repair enzyme may be purchased from AGI/Dermatics under the trade name Roxisomes® and has the INCI name Arabidopsis Thaliana extract. It may be present alone or in admixture with lecithin and water. This DNA repair enzyme is known to be effective in repairing 8-oxo-diGuanine base mutation damage.

Another type of DNA repair enzyme that may be used is one that is known to be effective in repairing 06-methyl guanine base mutation damage. It is sold by AGI/Dermatics under the tradename Adasomes®, and has the INCI name Lactobacillus ferment, which may be added to the composition of the invention by itself or in admixture with lecithin and water.

Another type of DNA repair enzyme that may be used is one that is known to be effective in repairing T-T dimers. The enzymes are present in mixtures of biological or botanical materials. Examples of such ingredients are sold by AGI/Dermatics under the tradenames Ultrasomes® or Photosomes®. Ultrasomes® comprises a mixture of Micrococcus lysate (an end product of the controlled lysis of various species of Micrococcus), lecithin, and water. Photosomes® comprises a mixture of plankton extract (which is the extract of marine biomass which includes one or more of the following organisms: thalassoplankton, green micro-algae, diatoms, greenish-blue and nitrogen-fixing seaweed), water, and lecithin.

Another type of DNA repair enzyme may be a component of various inactivated bacterial lysates such as Bifida lysate or Bifida ferment lysate, the latter a lysate from Bifido bacteria which contains the metabolic products and cytoplasmic fractions when Bifido bacteria are cultured, inactivated and then disintegrated. This material has the INCI name Bfida Ferment Lysate.

### Sunscreens

The compositions of the present invention may comprise one or more sunscreen actives or sunscreen agents. Examples of suitable sunscreen actives include oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10th Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof. All known sunscreen actives are considered to be within the scope of the present invention.

### Humectants

The composition may contain one or more humectants. If present, the humectants may range from about 0.1 to 75%, preferably from about 0.5 to 70%, more preferably from about 0.5 to 40%. Examples of suitable humectants include, without limiting, glycols, sugars, and the like. Suitable glycols are in monomeric or polymeric form and include polyethylene and polypropylene glycols such as PEG 4-10, which are polyethylene glycols having from 4 to 10 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol, glycerin, propylene glycol, or hexylene glycol.

### Botanical Extracts

It may be desirable to incorporate one more botanical extract into the composition. If present suggested ranges are from about 0.0001 to 20%, preferably from about 0.0005 to 15%, more preferably from about 0.001 to 10%. Suitable botanical extracts include, without limiting, extracts from plants (herbs, roots, flowers, fruits, seeds) such as flowers, fruits, vegetables, and so on, including yeast ferment extract, Padina Pavonica extract, Thermus Thermophilis ferment extract, Camelina Sativa seed oil, Boswellia Serrata extract, olive extract, Acacia Dealbata extract, Acer Saccharinum (sugar maple), Acidopholus, Acorus, Aesculus, Agaricus, Agave, Agrimonia, algae, aloe, citrus, Brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Microcystis Pyrifera, Pyrus Malus, Saxifraga Sarmentosa, Vitis Vinifera, Morus Nigra, Scutellaria Baicalensis, Anthemis Nobilis, Salvia Sclarea, Prunus Amygdalus, Rosmarinus Officianalis, Sapindus makurossi, Caesalpinia spinosa, Citrus Medica Limonum, Panax Ginseng, Siegesbeckia Orientalis, Mangifera Indicia, Fructus Mume, Psidium Guajava, Ascophyllum Nodosum, Centaurium erythrea, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Helianthus Annuus, Hordeum Vulgare, Cucumis Sativus, Evernia Prunastri, Evernia Furfuracea, Kola Acuminata, glycyrretinic acid, and mixtures thereof.

### Peptides

It may be desirable to incorporate one or more peptides into the composition. The term “peptide” refers to biomolecules having from about 2 to 20 amino acids connected by peptide bonds. Preferred ranges of the peptide present in the composition is from about 0.001 to 20%, preferably from about 0.005 to 15%, more preferably from about 0.01 to 10%. Preferred are biologically active peptides including those set forth in the C.T.F.A. International Cosmetic Ingredient Dictionary and Handbook, Eleventh Edition, 2006, page 2712. Such peptides include, but are not limited to the CTFA names: Acetyl Hexapeptide-1, 7, 8; Acetyl Pentapeptide-1, 2, 3, or 5; Acetyl Tripeptide-1; Acetyl Dipeptide-1 cetyl ester; Acetyl Glutamyl Heptapeptide-3; Acetyl Glutamyl Hexapeptide-6; Acetyl Monofluoropeptide-1; Heptapeptide-1, 2, or 3; Hexapeptide-1, 2, 3, 4, 5, 6, 7, 8, 9, 10, 11, 12, 13, or 14; Manganese Tripeptide-1; Myristoyl Hexapeptide-5, 12, or 13; Myristoyl Nonapeptide-2; Myristoyl Pentapeptide-4; Myristoyl Tetrapeptide-4 or 6; Myristoyl Tripeptide-4; Nisin, Nonapeptide-1 or 2; Oligopeptide-1, 2, 3, 4, 5, 6, 7, 8, 9 or 10; Palmitoyl Hexapeptide-14; Palmitoyl Pentapeptide-4; Palmitoyl Pentapeptide-4 or 5; Palmitoyl Tripeptide-1 or 5; Pentapeptide-1, 2, 3, 4, 5, or 6; Tetrapeptide-1, 2, 3, 4, 5, 6, or 7; Tripeptide-1, 2, 3, 4, or 5; or Palmitoyl Oligopeptides. All peptides that has cosmetic or dermatologic applications are considered to be within the scope of the present invention.

In one preferred embodiment, the composition comprises Acetyl Hexapeptide-8, having the trade name Argireline®.

### Oils

The composition may also comprise one or more oils in the form of natural, synthetic, or silicone oils. The term “oil” refers to an ingredient that is pourable at room temperature, e.g., 25° C. Oils may be volatile or non-volatile. The term “volatile” means that the oil has vapor pressure greater than about 2 mm of mercury at 20° C. The term “non-volatile” means that the oil has a vapor pressure of less than about 2 mm. of mercury at 20° C. If present, suggested ranges are from about 0.1 to 60%, preferably from about 0.5 to 45%.

Examples of volatile oils include volatile linear, cyclic, or branched silicones such as cyclopentasiloxane, cyclohexasiloxane (2 cst), hexamethyldisiloxane (0.65 cst, centistokes), octamethyltrisiloxane (1.0 cst), decamethyltetrasiloxane (1.5 cst), or dodecamethylpentasiloxane (2.0 cst); or branched volatile silicones such as methyl trimethicone (1.5 cst). Also suitable are volatile paraffinic hydrocarbons such as isododecane, isohexadecane, C11-14 alkanes, and mixtures thereof.

Non-volatile oils include linear silicones commonly referred to as dimethicone, phenyl substituted silicones such as phenyl dimethicone, phenyl trimethicone, trimethylsiloxy phenyldimethicone, cetyl dimethicone, perfluorodimethicone, phenethyl dimethicone, and the like. Non-volatile oils may also include esters or hydrocarbons. Esters include C1-10 alkyl esters of C1-20 carboxylic acids. One preferred type of ester is a fatty acid (C6-22) ester of a straight or branched chain saturated or unsaturated C1-22 alkyl. Examples include esters that have a low viscosity, e.g., ranging from 10-100 cst at room temperature. Examples of such esters include but are not limited to jojoba esters. Other non-volatile oils include sterols such as phytosterols, phytosphingosine, and similar plant sterols.

### Thickeners

Suitable thickeners may be incorporated into the composition. Suitable thickeners may be present in ranges are from about 0.0001-45%, preferably from about 0.0005-40%.

Examples of thickeners include animal, vegetable, mineral, silicone, or synthetic waxes which may have melting points ranging from about 30 to 150° C. including, but not limited to waxes made by Fischer-Tropsch synthesis, such as polyethylene or synthetic wax or various vegetable waxes such as bayberry, candelilla, ozokerite, acacia, beeswax, ceresin, cetyl esters, flower wax, citrus wax, carnauba wax, jojoba wax, Japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax or fatty acids or fatty alcohols, including esters thereof, such as hydroxystearic acids (for example 12-hydroxy stearic acid), tristearin, tribehenin, and so on.

Also, suitable thickening agents such as silica, silicates, silica silylate, and alkali metal or alkaline earth metal derivatives thereof may be utilized in the composition. These silicas and silicates are generally found in the particulate form and may include silica, silica silylate, magnesium aluminum silicate, and the like.

Silicone elastomers may also be used as thickening agents. Such elastomers include those formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha-omega diene or by condensation-curing organopolysiloxane compositions in the presence of an organotin compound or a titanate ester using a condensation reaction between a hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene, and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals are preferred. The molecular structure of this component may be straight-chain, branched straight-chain, cyclic, or a network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in powder form or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Corning's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the trade names KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK.

Polysaccharides may be suitable aqueous phase thickening agents. Examples of such polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are different types of synthetic polymeric thickeners. One type includes acrylic polymeric thickeners comprised of monomers A and B wherein A is selected from the group consisting of acrylic acid, methacrylic acid, and mixtures thereof, and B is selected from the group consisting of a C1-22 alkyl acrylate, a C1-22 alky methacrylate, and mixtures thereof are suitable. Acrylic polymer solutions include those sold by Seppic, Inc., under the tradename Sepigel® or those sold under the tradename Aristoflex®.

Also suitable are acrylic polymeric thickeners that are copolymer of A, B, and C monomers wherein A and B are as defined above, and C has the general formula:

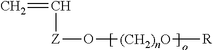

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate-based anionic amphiphilic polymers containing at least one hydrophilic unit and at least one allyl ether unit containing a fatty chain. Preferred are those where the hydrophilic unit contains an ethylenically unsaturated anionic monomer, more specifically a vinyl carboxylic acid such as acrylic acid, methacrylic acid, or mixtures thereof, and where the allyl ether unit containing a fatty chain corresponds to the monomer of the formula:

CH₂═CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl, and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C18) radical. Anionic amphiphilic polymers of this type are described and prepared in U.S. Pat. Nos. 4,677,152 and 4,702,844. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance, diallyl phthalate, allyl (meth)acrylate, divinylbenzene, (poly)ethylene glycol dimethacrylate and methylenebisacrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide, and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate-based polymers where one or more of the acrylic groups may have substituted long-chain alkyl (such as 6-40, 10-30, and the like) groups, for example, acrylates/C10-30 alkyl acrylate crosspolymer which is a copolymer of C10-30 alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames and have the CTFA name carbomer.

One particularly suitable type of aqueous phase thickening agent are acrylate-based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in a mixture containing caprylic/capric triglyceride, trilaureth-4, and polyglyceryl-2 sesquiisostearate; or Aristoflex HMB which is ammonium acryloyldimethyltaurate/beheneth-25 methacrylate cross polymer, and the like.

Also, suitable as thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CTFA names polyglycerin-20, polyglycerin-40, and the like.

### Surfactants

The compositions of the invention may contain one or more surfactants. This is particularly desirable when the composition is in the form of an aqueous gel or emulsion. If present, the surfactant may range from about 0.001 to 50%, preferably from about 0.005 to 40%, more preferably from about 0.01 to 35% by weight of the total composition. Suitable surfactants may be silicone or organic, nonionic, anionic, amphoteric, or zwitterionic. Such surfactants include, but are not limited to, those set forth herein and are well known in the art.

### Vitamins and Antioxidants

The compositions of the invention may contain vitamins and/or coenzymes, as well as antioxidants. The composition may include vitamins and/coenzymes in amounts ranging from about 0.001-10%, preferably 0.01-8%, more preferably 0.05-5% by weight of the total composition is suggested. Suitable vitamins include ascorbic acid and derivatives thereof such as ascorbyl palmitate, tetrahexydecyl ascorbate, and the B vitamins such as thiamine, riboflavin, pyridoxin, as well as coenzymes such as thiamine pyrophoshate, flavin adenin dinucleotide, folic acid, pyridoxal phosphate, tetrahydrofolic acid, and so on. Also, Vitamin A and the derivatives thereof are suitable. Examples are retinyl palmitate, retinol. retinoic acid, as well as Vitamin A in the form of beta carotene. Also, suitable is Vitamin E and derivatives thereof such as Vitamin E acetate, nicotinate, or other esters thereof. In addition, Vitamins D and K are suitable.

Suitable antioxidants are ingredients, which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like. In one preferred embodiment, the composition is free of parabens.

### Particulate Materials

The compositions of the invention may contain particulate materials in the form of pigments, inert particulates, or mixtures thereof. Such particulate material may be present in ranges from about 0.01-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.

A. Powders

The particulate matter may be colored or non-colored (for example, white) non-pigmented powders. Suitable non-pigmented powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above-mentioned powders may be surface treated with lecithin, amino acids, mineral oil, silicone, or various other agents either alone or in combination, which coat the powder surface and renders the particles more lipophilic in nature.

B. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

### Carrier

The compositions comprise a dermatologically or cosmetically acceptable carrier for the skincare active materials. “Dermatologically/cosmetically acceptable,” as used herein, means that the compositions or components described are suitable for use in contact with human keratinous tissue without undue toxicity, incompatibility, instability, allergic response, and the like. The compositions may comprise from about 50% to about 99.99%, alternatively from about 60% to about 99.9%, alternatively from about 70% to about 98%, and alternatively from about 80% to about 95% of the composition.

The carrier can be a wide variety of types, non-limiting examples of which include solutions, dispersions, emulsions, and combinations thereof. “Emulsions” generally contain an aqueous phase and an oil phase. The oils may be derived from animals, plants, or petroleum, may be natural or synthetic, and may include silicone oils. Emulsion carriers include but are not limited to oil-in-water, water-in-oil, and water-in-oil-in-water emulsions. In one embodiment, the carrier comprises an oil-in-water emulsion, a water-in-oil emulsion a silicone-in-water emulsion, and/or a water-in-silicone emulsion. The emulsions may comprise from about 0.01% to about 10%, and alternatively from about 0.1% to about 5%, of a nonionic, anionic, or cationic emulsifier, and combinations thereof. Suitable emulsifiers are disclosed in, for example, U.S. Pat. Nos. 3,755,560, 4,421,769, and McCutcheon's Detergents and Emulsifiers, North American Edition, pages 317-324 (1986).

The invention will be further described in connection with the following examples which are set forth for purposes of illustration only.

### EXPERIMENTS

A non-limiting skin exemplary penetration study was performed to evaluate the effects of the composition disclosed herein as example 1 comprising active ingredients of varied hydrophilicity. The formulation used in the experiment is shown in Table 1.

### Example 1

TABLE 1
Exemplary Formulation

| Formula # | Type of Chopped Fibers | Actives |
|---|---|---|
| Formula 1 | No fiber, control crème | 1.8% of |
| Formula 2 | 1% of Micro Cellulose Fiber | AA2G, 0.1% |
| Formula 3 | 1% of Cellulose Acetate Fiber | of Caffeine |
| Formula 4 | 1% of Micro Cellulose Fiber + 1% of | and 0.18% of |
| | Cellulose Acetate Fiber | hydrophobic |
| | | active |

All formula's shown in Table 1 include 1.8% of AA2G, 0.1% of Caffeine, and 0.18% of the hydrophobic active. The formulation was prepared by mixing the active ingredient and chopped polymeric nanofibers or microfibers. Cellulose acetate microfiber or cellulose acetate nanofiber was utilized according to the present invention and the experiment was conducted. The study evaluated the penetration efficacy of the active ingredient in presence of chopped nanofibers in the composition. The delivery and penetration efficacy of actives with different hydrophilicities in formulations was studied. In particular, the delivery efficacy of caffeine, hydrophobic active, and ascorbic acid 2-glucoside (AA2G) from a formula containing hydrophobic nanofibers was analyzed. The active ingredients studied cover hydrophilic to very hydrophobic ranges. Two types of polymer nanofibers were used. To better disperse the polymer nanofiber, the nanofibers were pre-cut into a few cm in length. Such kind of nanofiber was referred to as “chopped fibers”.

The data indicated that in the presence of cellulose acetate chopped fibers, the delivery of AA2G into the skin was enhanced in the initial 6 hours after loading. A penetration study was then performed using the formulation without fiber as a control. The penetration efficacies of caffeine and AA2G were then compared.

### Skin Penetration Study (Franz Cell) Using MatTek Skin

In a 6-well plate, MatTek 300 skin (a skin modeling constructed by live cells, obtained from MatTek) was placed in well with 2.00 mL of incubation medium (also provide by MatTek). To the top of the skin, 400 ul of the sample was loaded. The skin was then incubated at 37° C. At 3 hours and 6 hours, the incubation medium was collected, and another 2.00 mL of medium was added into the well. The skin was then incubated until 24 hours. Finally, the medium was collected at 24 hours' time point. The collected mediums were filtered through a 0.45 um PTFE syringe filter into HPLC vials. The samples were then submitted to the UPLC study.

### UPLC Analysis

Waters ACQUITY H UPLC was used to analyze the collected samples from the penetration study (Franz Cell study). The UPLC parameters are shown in Table 2.

TABLE 2
UPLC study parameters

| | | Hydrophobic | AA2G |
|---|---|---|---|
| | Caffeine | active | (Hydrophilic active) |
| Column | XSelect HSS T3 2.5 um 3.0 * 50 mm Column XP | | |
| Mobile Phase | 95 % DI water, 5% of 0.1% Formic | | 0.1% Formic Acid in DI water |
| | Acid in Acetonitrile | | |
| Flow rate | 0.80 mL/min | | 0.80 mL/min |
| Column Temp (° C.) | 40 | | 40 |
| Injection volume (ul) | 2 | | 2 |
| Detection | 254 | 280 | 254 |
| wavelength (nm) | | | |

The calibration curves for each active were plotted using standards.

### Skin PAMPA Study

In vitro delivery of active ingredients in a formulation was evaluated using a skin PAMPA study. Active ingredients and pulverized thin fibers were added to the formulation and tested for permeation using PAMPA SC membrane for polar compounds. The following samples were prepared. Formulations are provided in Table 3 in percentages.
Formula 1 as shown in Table 3.
Formula 2: Formula 1 and 1% of microcrystalline cellulose (9 μm).
Formula 3: Formula 1 and the maximum amount of 1% dispersion (cellulose acetate fibers in water).
Formula 4: Formula 1 and 1% of microcrystalline cellulose (9 μm) and the maximum amount of 1% dispersion (cellulose acetate fibers in water).

TABLE 3

| Ingredient Name | Formula 1 | Formula 2 | Formula 3 | Formula 4 |
|---|---|---|---|---|
| Microcrystalline cellulose | | 1 | | 1 |
| Cellulose acetate fibers in water | 40 | 40 | 40 | 40 |
| Cyclopentaxsiloxane | 13 | 13 | 13 | 13 |
| Dimethicone | 12 | 12 | 12 | 12 |
| Polysilicone 11 | 7.5 | 7.5 | 7.5 | 7.5 |
| isododecane | 7 | 7 | 7 | 7 |
| butylene glycol | 6 | 6 | 6 | 6 |
| peg10 dimethicone | 1.5 | 1.5 | 1.5 | 1.5 |
| peg 6 | 1 | 1 | 1 | 1 |
| ammonium acryloyldimethyltaurate/vp | 0.6 | 0.6 | 0.6 | 0.6 |
| copol | | | | |
| acrylamide/sodiumacryloyldimethyltaurate | 0.6 | 0.6 | 0.6 | 0.6 |
| polysorbate 20 | 0.5 | 0.5 | 0.5 | 0.5 |
| Phenoxyethanol | 0.4 | 0.4 | 1.2 | 1.2 |
| caprylyl glycol | 0.2 | 0.2 | 0.2 | 0.2 |
| caffeine | 0.2 | 0.2 | 0.2 | 0.2 |
| Hydrophobic active | 0.18 | 0.18 | 0.18 | 0.18 |
| asorbyl glucoside | 1.8 | 1.8 | 1.8 | 1.8 |
| tromethamine | 0.65 | 0.65 | 0.65 | 0.65 |
| Purified water | qs100 | qs100 | qs 100 | qs100 |

### Penetration of AA2G in the Presence of Chopped Nanofibers

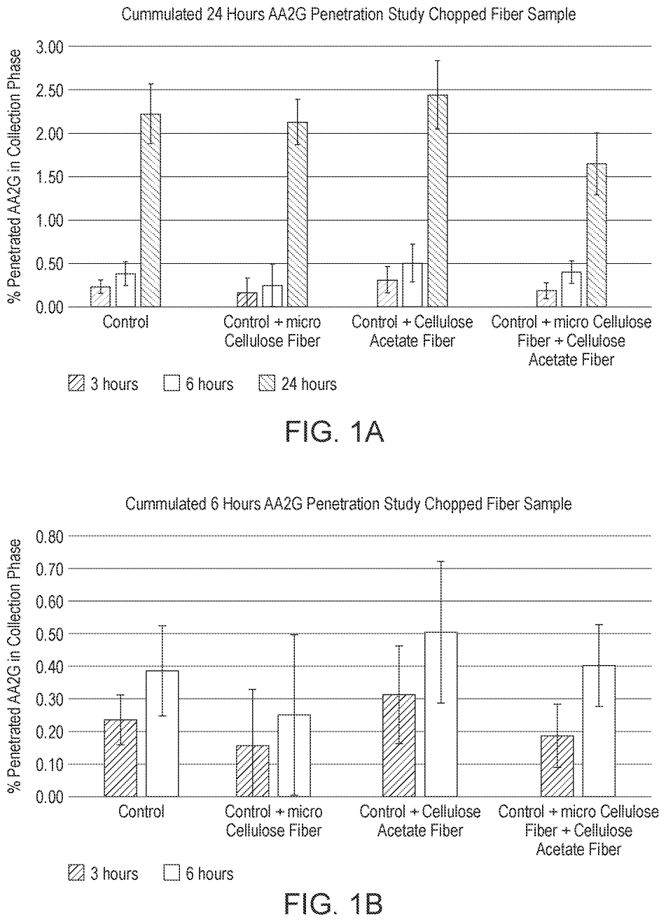

AA2G is very hydrophilic skincare active. The percentage of the penetration of the AA2G from the formula was shown in FIGS. 1A and 1B as obtained from the skin model (Franz Cell) penetration study. At three hours and six hours time points, the formula containing cellulose acetate chopped fibers showed higher penetration efficacy than the control. At three hours time point, the enhancement was 33% and at six hours' time point, the enhancement was 30%. At twenty-four hours, this difference was not significant, that is less than 10% indicating that the chopped fiber may enhance the penetration of AA2G in a specific duration, i.e., the early stage after the application.

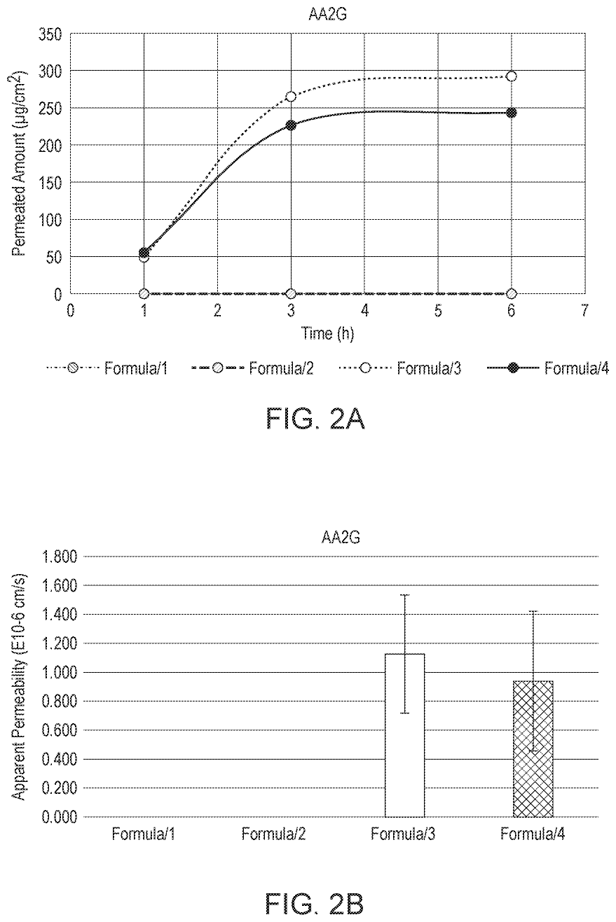

FIGS. 2A and 2B show the permeability of AA2G in the skin PAMPA study.

FIG. 2A shows the results of the skin PAMPA study and indicates that the control with no fiber (Formula 1) showed no penetration of active ingredient as obtained in the PAMPA study. However, cellulose acetate (chopped nanofiber) with active showed high penetration of the activity followed by cellulose acetate microfiber and nanofiber combination in FIG. 2A.

FIG. 2B shows the permeability of AA2G in skin PAMPA study indicating that delivery of AA2G was very low in control and Vivapure (1% microcrystalline cellulose (9 μm)) while the delivery and permeability of AA2G was found to be substantial and increased in the formulation having the chopped fibers (Formula 3: 1% dispersion comprising cellulose acetate fibers in water and Formula 4: 1% microcrystalline cellulose and dispersion comprising 1% cellulose acetate fibers in water).

Based on the skin model penetration (FIGS. 1A and 1B), Franz cell study, at 3 hours the enhancement was 33% and at 6 hours the enhancement was 30%. At 24 hours this difference was not as significant (less than 10%). This indicates that the chopped fiber may enhance the penetration of AA2G in a specific duration, i.e., the early stage after the application. We analyzed the statistical significance of the percentage of penetration of AA2G between the control and the formula containing Cellulose Acetate fibers. The p value is greater than 0.05. Combined with the PAMPA study result (shown in FIG. 2A), the data of this study showed that the formula having cellulose acetate chopped fibers enhanced the penetration of the AA2G. The permeated amount as shown in FIG. 2B is about 0 to 300 μg/cm2.

FIG. 2C shows the results of the skin model penetration (Franz cell) study from 3 hours' time point. FIG. 2D shows comparative results of the six hours penetration of AA2G between skin PAMPA study and skin penetration study results.

### Example 2

### Penetration of Caffeine in the Presence of Chopped Nanofibers

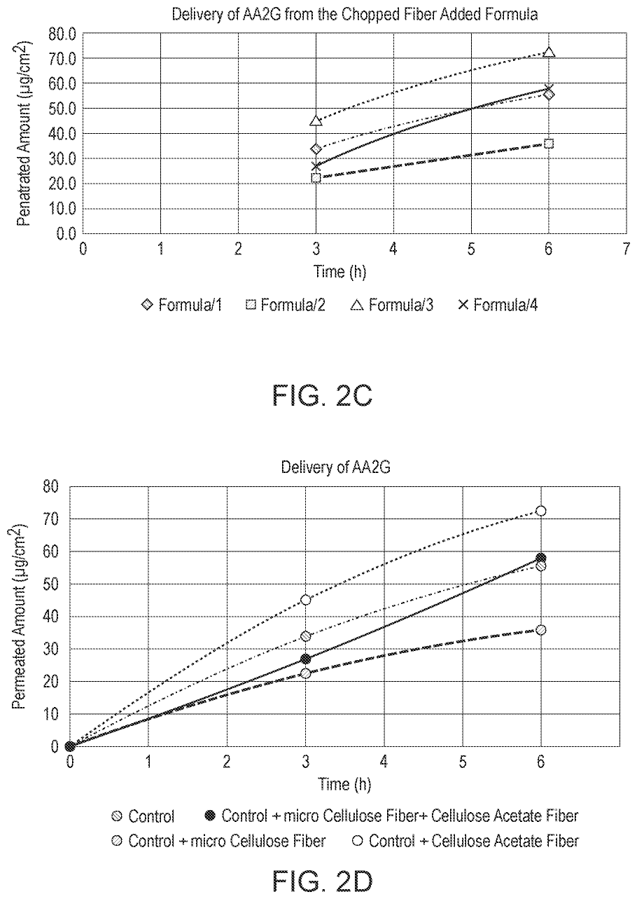

Caffeine is an active that is both water and oil soluble. FIG. 3 shows penetration study of caffeine in chopped fibers formula in 24 hours. Based on the data shown in FIG. 3, the penetration of caffeine was not significantly enhanced by the addition of chopped fiber (<10%) at six-hour time point.

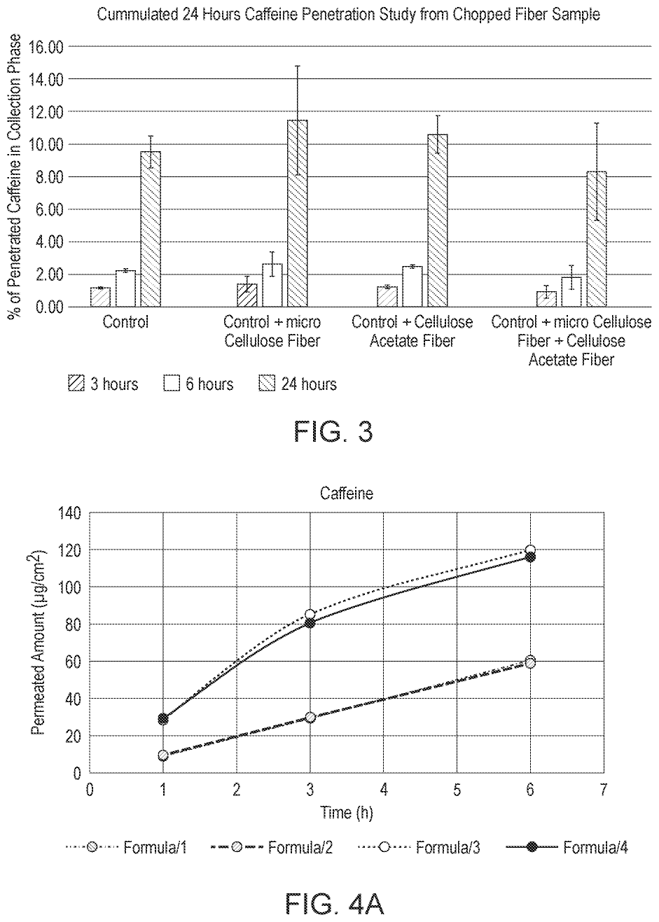

Results of the skin PAMPA study for caffeine are shown in FIGS. 4A and 4B. FIG. 4A shows the results of PAMPA study results while FIG. 4B shows the delivery of caffeine in a composition comprising chopped fibers causing a doubling of permeation in the presence of the chopped fibers compared to the control. FIG. 4B is the result of the skin PAMPA study.

FIG. 4A shows the six hours penetration study data of caffeine obtained by skin PAMPA showed that cellulose acetate nanofiber has slightly increased permeating amount of active ingredient followed by the combination of microfibers and nanofibers. This result is consistent with the results of the penetration study of AA2G.

FIG. 4C shows the results of skin model penetration (Franz Cell) of the delivery of caffeine from the chopped fiber added formula.

The results of FIG. 3 at the six-hour time point may be explained by the data obtained from the skin PAMPA study. Because of the use of an artificial skin layer in the skin PAMPA studies, which is made by polymer and variation to mimic the live skin layer may exist. Also, the percentage % penetration of caffeine was greater than that of AA2G, consistent with the previous penetration studies using other crème and in silico modelling. [[connect to benefits of claim]]

### Example 3

### Penetration of Hydrophobic Active in the Presence of Chopped Nanofibers

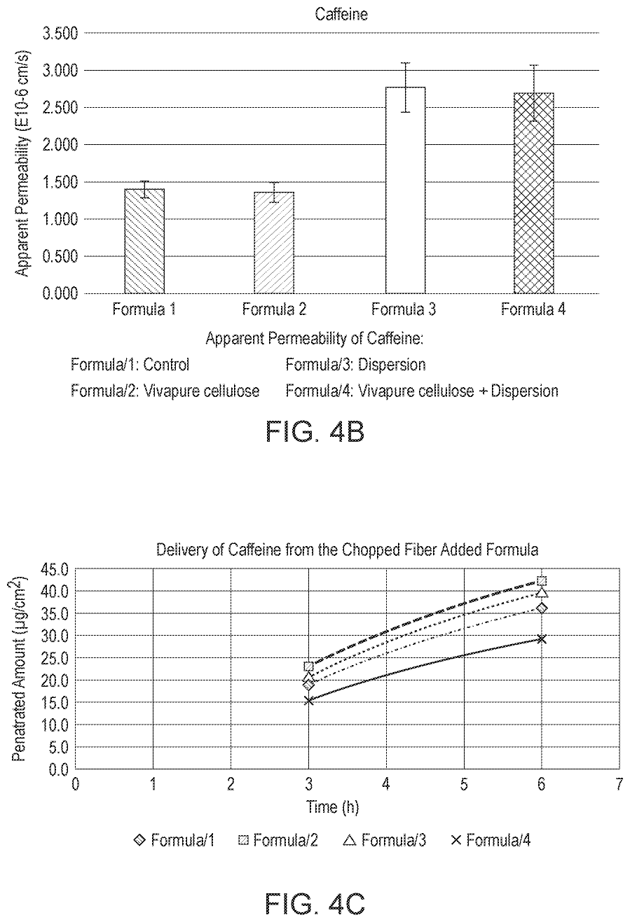

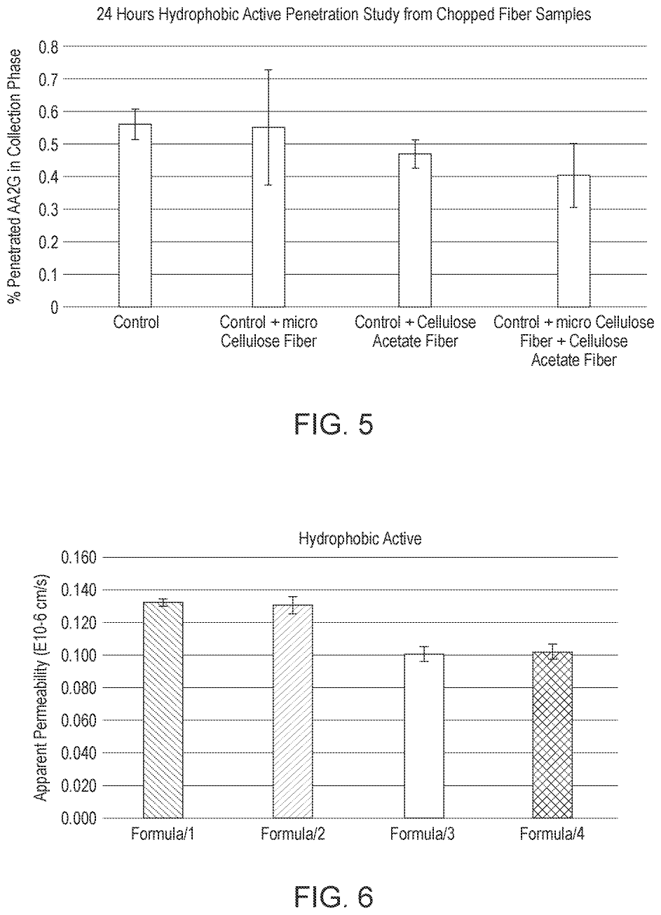

A hydrophobic active was utilized in the study. It is not soluble in water. FIG. 5 shows the results of penetration of the hydrophobic active and chopped fibers in 24 hours via skin penetration study (Franz Cell). Based on the data shown in FIG. 5, the penetration of the hydrophobic active was not significantly enhanced by the addition of chopped fiber. This is consistent with the data obtained by the skin PAMPA study as shown in FIG. 6.

Applicants of the present invention unexpectedly and surprisingly achieved the cosmetic benefit of improving the skin's appearance using the composition. The composition provided significant benefits, showing a reduction in the dilation of the veins, bags under the eyes, dark circles under the eyes, and swelling around the eyes, fine lines and wrinkles, loss of elasticity, wrinkles, loss of stiffness, loss of uniformity of color or tone, rough surface texture, age spots, and a decrease in moisture content in the skin around the eyes and in the facial skin. Additional benefits obtained by the composition include, without limiting, the use of the composition as an antioxidant, collagen booster, as well as, lightening of dark spots, smoothening of wrinkles, promotion of healing, and/or reduction of scars.

The experiments and analysis of the results indicate an enhancement of penetration efficacy of a variety of actives in the presence of chopped fiber via both skin penetration study and PAMPA study. Modelling skins consisted of live cells and they were used to better mimic the permeability of the skin. The cellulose acetate chopped fibers enhanced the penetration of AA2G, and slightly enhanced the penetration of caffeine. There was a hydrophobicity dependence in the enhancement effect. The microfibers of cellulose acetate (dispersed) did not show a significant enhancement in the penetration of all the selected actives. However, dispersed/chopped nanofibers showed enhanced penetration of actives. Such a result was consistent with the result obtained by the skin PAMPA study.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the scope of the invention. It is therefore intended to cover in the appended claims all such changes and modifications that are within the scope of this invention.

What is claimed is:

1. A method of reducing skin defects of a subject's skin, comprising topically applying a composition comprising electrospun hydrophobic fibers and at least one active ingredient, wherein the fibers are dispersed in the composition.

2. The method of claim 1, wherein the composition further comprises one or more cosmetically acceptable carriers.

3. The method of claim 1, wherein the method further improves the visual appearance of the subject's skin.

4. The method of claim 1, wherein the method further improves dilation of veins, bags under the eyes, dark circles under the eyes, swelling around the eyes, fine lines, wrinkles, loss of elasticity, loss of stiffness, loss of uniformity of color, tone, rough surface or texture, age spots, or moisture content in the skin.

5. The method of claim 1, wherein the composition is applied at least once a day.

6. The method of claim 1, wherein the composition is applied in the morning and prior to retiring to bed.

7. The method of claim 1, wherein the topical application is a treatment regimen.

8. The treatment regimen of claim 7, wherein the treatment regimen further comprises applying at least one additional cosmetic composition.
